(19) United States
(12) Patent Application Publication
(10) Pub. No.: US 2026/0060579 A1
(43) Pub. Date: Mar. 5, 2026

(54) HUMAN FUNCTIONAL STATE DETECTOR

(71) Applicant: Harman International Industries, Incorporated, Stamford, CT (US)

(72) Inventors: Andrey Viktorovich Filimonov, Novi, MI (US); Ivan Sergeevich Shishalov, Yeravan (AM); Anastasiya Vladimirovna Bakhchina, Nizhny Novgorod (RU); Anastasiya Sergeevna Filatova, Balakhna (RU); Grigoriy Sergeevich Radchenko, Nizhny Novgorod (RU); Burova Anzhela Grigorevna, Yeravan (AM); Evgeny Pavlovich Burashnikov, Yerevan (AM); Mikhail Sergeevich Kleshnin, Nizhny Novgorod (RU); Daniil Igorevich Konovalov, Yerevan (AM); Mikhail Sergeevich Sotnikov, Nizhny Novgorod (RU); Anton Sergeevich Devyatkin, Nizhny Novgorod (RU); Sergey Konstantinovich Arzamaskin, Nizhny Novgorod (RU); Anton Yakimov, Nizhny Novgorod, (RU); Mariya Yurievna Nevaykina, Nizhny Novgorod (RU)

(21) Appl. No.: 19/132,767

(22) PCT Filed: Jun. 23, 2023
(86) PCT No.: PCT/US2023/069009

§ 371 Date: May 23, 2025

(30) Foreign Application Priority Data
Nov. 23, 2022 (RU) 2022130395

(51) Int. Cl. A61B 5/16; A61B 5/00; A61B 5/18; B60Q 9/00

(52) U.S. Cl. CPC A61B 5/165; A61B 5/0077; A61B 5/18; A61B 5/6893; A61B 5/746; B60Q 9/00; A61B 2503/22

(57) ABSTRACT

According to one aspect of the present disclosure, there is provided a computer-implemented method for detecting human functional states, the method comprising: obtaining one or more signals from one or more sensors, wherein the signals are representative of a plurality of physiological parameters of the user; determining a plurality of conditions associated with the user based on values of the physiological parameters; determining a functional state of the user based on the conditions; and generating an output signal to indicate the functional state of the user.

### CROSS REFERENCE TO RELATED APPLICATIONS

The present application claims priority to Russian Application No. 2022130395, entitled “HUMAN FUNCTIONAL STATE DETECTOR”, and filed on Nov. 23, 2022. The entire contents of the above-listed application are hereby incorporated by reference for all purposes.

### FIELD

The present disclosure relates to methods and systems for detecting human functional states.

### BACKGROUND

Identifying mental and physical states of a human is an important aspect of the development of human machine interfaces (HMI). The ability to identify a human's state reliably and without influencing the human's behaviour can be used to solve various industrial problems. Applications may range from monitoring drivers and workers to improving the interaction in computer games and social media. In such applications, the knowledge of human states can be used to mitigate risks and improve efficiency.

There are different approaches to identifying mental human states. For example, it is known to monitor a driver in car to detect if s/he is falling asleep. To this end, a camera arranged on a dashboard (or elsewhere) records images of the driver's face. The images are processed to determine physiological parameters such as the closure of the eyes or the frequency of blinks. From these parameters, it is possible to determine if the driver is in a potentially dangerous drowsy state that could lead to her/him falling asleep and causing an accident.

It is also known to use machine learning and/or artificial neural networks in advertising and marketing to identify human emotions from facial video images, e.g., to evaluate the effect of a given advertisement. Further, it is known to use a wearable (smart watch, fitness tracker) with a photoplethysmograph (a green LED and a photo resistor of the same wavelength) to record heartbeat rates, and to analyze recorded sequences to detect the stress level of the gadget wearer.

These and other state-of-the-art systems are designed to match a specific task. In particular, these systems identify the presence or level of a single condition, such as sleepiness or stress, based on one or more biosignal(s). In the broadest sense, a biosignal describes or represents an activity, state or function of a biological object. For example, a facial image can be used to identify a level of eye closure of a human. In this example, the biosignal can be a sequence of digital pictures of the human face which can be processed to derive the eye closure level. Another example is the photodetector output of a photoplethysmograph, which is proportional to the intensity of the green light beam reflected from the skin, which in turn is proportional to the oxygen concentration in the blood passing through the skin vessels. A further example is the microdisplacement of a human back reflecting respiratory patterns.

In order to identify a human state, the biosignal is input into a state detector. A state detector is a set of mathematical operations and signal transformations that converts the input signal, or part thereof, into a detector output. The state detector output is a number or logical value (1/0, Yes/No, True/False) associated with a particular state or state level. For example, for sleepiness detectors, 0 indicates “not sleepy” (wakeful), and 1 indicates “drowsy” (falling asleep). In case of a numerical output between 0 and 1, the value can be associated with a level of sleepiness. If the detector output is 0.1 at one time point (or for one person), and 0.2 at another time point (or for another person), this indicates that a more sleepy state is observed at the second time point (or for the other person).

A typical process performed by a system including a single-component state detector as described above is shown in FIG. 1. Such system may be expanded to include two or more single-component state detectors, each dedicated to detect a different component of the human state, such as stress and drowsiness. However, the human overall state at each point in time includes or reflects numerous (all) aspects of the person's behavior and functioning. While the separation of the overall state into distinct components makes sense methodologically, to simplify research, it may lead to results that do not represent the full picture. In addition, biosignal sensors will always have some unavoidable noise level that affects the accuracy of state detection. Typical accuracy is between 70% and 90% depending on the task. This means that prior art systems can make wrong detections of the human state and may not be sufficiently reliable.

In the automotive domain, the detection of biosignals is usually based on sensors that are remote from the driver. Signals from such remote sensors have a higher noise level than signals from contact sensors. In addition, the automotive environment is innately noisy. There may be mechanical or environmental noise. Image sensors may be exposed to changing light conditions. Thus, in the automotive domain in particular, it is a challenge to detect human functional states with sufficient accuracy.

The present disclosure aims to improve the accuracy and reliability of human functional state detection.

### SUMMARY

The present disclosure provides a method and system for identifying multiple human state components simultaneously and consistently. Interdependencies between multiple individual components are taken into account, thereby improving the accuracy and reliability of human functional state detection. This enables responding to detected human functional states with greater confidence, for example in the context of intervening in the operation of a vehicle when a critical state of the driver is detected.

The present disclosure recognizes that physiology imposes limitations on possible combinations of certain components of a person's state. For example, it is unlikely for someone to experience a high level of stress and a high level of sleepiness at the same time. On the other hand, happiness can occur at the same time as alertness. Thus, the detection of a certain component may make the presence of another component more or less likely. By defining such interrelations, the detection of individual components can be refined. This, in turn, improves the accuracy of determining an overall human functional state.

To achieve these and other desired effects, according to one aspect of the present disclosure, there is provided a computer-implemented method for detecting functional states of a user, the method including: obtaining one or more signals from one or more sensors, wherein the signals are representative of a plurality of physiological parameters of the user; determining a plurality of conditions associated with the user based on values of the physiological parameters; determining a functional state of the user based on a combination of the conditions; and generating an output signal to indicate the functional state of the user.

The method is particularly suited for implementation in a vehicle. However, the present disclosure is not limited in this regard, and the method may also be implemented in other systems in which a functional state of a user is to be detected.

The signals may be video signals obtained by one or more cameras arranged in a vehicle and directed at the user. For example, the video signals may be obtained from light reflected from the skin of the user onto an image sensor of the cameras. Changes in the intensity of the light correspond to changes in blood volume, from which heartbeat parameters may be derived. Accordingly, in one embodiment, the one or more parameters include one or more heartbeat parameters, in particular a heartbeat rate, a heartbeat rate variability, and/or a heart inter-beat interval.

Alternatively, or in addition, one or more cameras may be arranged to capture images of one or both of the eyes of a user. The eyes of the user and parameters relating to the eyes such as eye or eyelid positions and movements thereof may be identified using known feature detection methods. Accordingly, in an embodiment, the one or more physiological parameters include one or more eye parameters, in particular an eye movement, an eyelid movement, an eye position, an eyelid position, and/or an eye gaze.

Using heartbeat features and eye parameters can be advantageously combined to determine different components or aspects of the user's state. The user's state may include different physiological or psychological components, or a combination thereof. Such different components of the user's state are also referred to as “conditions” herein, while the user's state is also referred to as the user's “overall” state. Detected individual components may be combined to derive the user's state. For example, an increased heartbeat rate and rapid eye movements (saccades) may both point towards the same state, namely a high level of stress. Combining the detection of an increased heartbeat rate and saccades can thus be used to more reliably determine the overall state of the user.

The state of the user is indicative of the user's fitness to control, in particular drive the vehicle. In an embodiment, the user's state is indicative of one of the following states: fit to drive, conditionally fit to drive, and unfit to drive. If the user's state is determined to be “fit to drive”, no responsive action is taken. If the user's state is determined to be “conditionally fit to drive”, appropriate action can be taken. For example, a message may be displayed to recommend slowing down the vehicle or taking a break. If the user's state is determined to be “unfit to drive”, more drastic actions may be taken. For example, a loud warning signal may be generated, or the vehicle may be slowed down. However, the present disclosure is not limited to these states and actions. For example, there may be different levels of “conditionally fit to drive” each associated with respective actions. Also, the present disclosure is intended to encompass applications in vehicles other than cars, e.g. planes.

The state of the user may be associated with different components or conditions. For example, the user's state may be associated with different levels of stress, cognitive demand or drowsiness. Individual conditions may be correlated or interdependent with other individual conditions. They may supplement one another or be mutually exclusive. For example, when a user is exposed to an increased level of cognitive demand, s/he may at the same time experience an increased level of stress. The knowledge of one or more conditions can thus be used to more reliably detect other individual conditions. Accordingly, in an embodiment of the present disclosure, the method includes determining at least one of the conditions based on at least one other of the conditions. In particular, the method may include determining at least one of the conditions based on a combination or pattern of other conditions.

On the other hand, a high level of stress is usually not found when the user is drowsy. In terms of physiological parameters, a high heartbeat rate usually does not occur when the user has half-closed eyes over a certain period of time. Accordingly, the interdependency of individual conditions may be by way of mutual exclusivity. Detecting such mutually exclusive conditions enables identifying potential errors caused, for example, by noisy image signals due to insufficient light. Thus, taking into a combination of conditions enables eliminating errors and determining the user's overall state more reliably.

The cognitive load of a user may include a cognitive load related to controlling the vehicle. For example, if the user's eye gaze indicates that s/he is looking at a road ahead, the current cognitive load can be determined to relate to driving. On the other hand, the user's eye gaze may indicate that s/he is looking at a mobile telephone, and the current cognitive load can be determined to be unrelated to driving. Differentiating between such conditions enables determining different categories of the “conditionally fit to drive”state, for example.

In an embodiment, determining the state of the user includes selecting one of a plurality of predetermined states, wherein each of the plurality of predetermined states is associated with a respective combination of conditions. For example, the “fit to drive” state may be determined if the conditions “no stress” (indicated by a normal heart rate, for example) and “attentive” (indicated by an eye gaze directed to the road ahead, for example) are both present at the same time. If, on the other hand, the conditions are contradictory, one of the conditions may be determined to be more likely, for example based on stored information. In such case, the more likely condition is selected for determining the user's state, while the other condition is discarded. Alternatively, determining the user's state may be suspended until the detection of contradictory conditions is over. Also, an error signal may be generated.

In an embodiment, several conditions are taken into account, e.g. three or more, and the method includes determining that some, e.g. at least two of the conditions are mutually exclusive, and discarding one or all of the mutually exclusive conditions when determining the state of the user. This embodiment enables establishing a desired level of confidence by requiring the presence of a minimum number of consistent conditions as a prerequisite for determining the user's overall state. However, the present disclosure is not limited to a particular number of conditions. Also, the level of confidence may be adjusted depending on the user's state to be determined. For example, the “fit to drive” state may require a higher number of consistent conditions than the “conditionally fit to drive” state. In another example, the “unfit to drive” state may require the absence of any combination of inconsistent conditions.

In an embodiment, generating an output signal to indicate the state of the user includes one or more of alerting the user of the vehicle, controlling the vehicle, in particular adjusting a user interface in the vehicle or adjusting one or more vehicle control systems, and communicating the state of the user to another vehicle or an external server or control station. Thereby, the safety in operating the vehicle may be improved.

According to another aspect of the present disclosure, there is provided a system for detecting functional states of a user, the system including one or more sensors to be arranged in the vehicle to detect one or more signals, wherein the signals are representative of a plurality of physiological parameters of the user, and at least one processing device configured to determine a plurality of conditions associated with the user based on values of the physiological parameters, to determine a functional state of the user based on a combination of the conditions, and to generate an output signal to indicate the functional state of the user. Similar to the method described above, the system enables improving the reliability and accuracy of determining the user's overall state, for example the user's fitness to operate (drive) a vehicle.

The system may be configured to implement any of the operations, functions and advantages described above. In particular, the at least one processing device may be configured to determine the state of the user by selecting one of a plurality of predetermined states, wherein each of the plurality of predetermined states is associated with a respective combination of conditions. Also, the system may be configured to determine the user's state based on several, e.g. three or more conditions, wherein the processing device is configured to determine that some, e.g. at least two of the conditions are mutually exclusive, and to discard one or all of the mutually exclusive conditions when determining the state of the user.

The system may be an in-vehicle system and include one or more in-vehicle sensors to generate the signals that are processed to determine the user's state. The sensors may include one or more image sensors, in particular a combination of different cameras, e.g. RGB and infrared cameras, and/or cameras arranged at different locations of the vehicles. In this way, the issue of variable light conditions may be addressed and the accuracy of image processing and feature detection improved.

In an embodiment, the at least one processing device includes at least one processing device arranged at a remote server or control station. Accordingly, the method of the present disclosure can be partially or wholly implemented remotely from the vehicle, thereby reducing the processing load in the vehicle.

### BRIEF DESCRIPTION OF THE DRAWINGS

The features, objects, and advantages of the present disclosure will become more apparent from the detailed description set forth below when taken in conjunction with the drawings in which like reference numerals refer to the same or similar elements.

FIG. 1 illustrates a conventional approach to detecting human functional states;

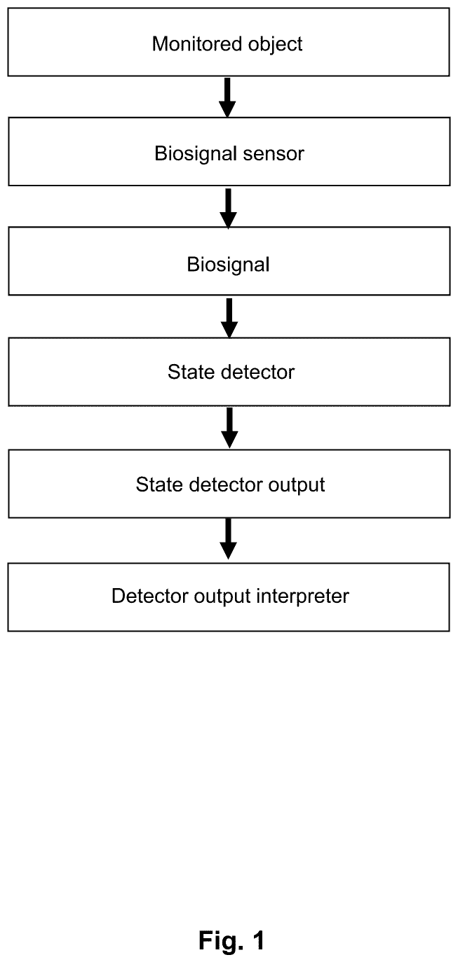

FIG. 2 schematically illustrates a system for determining a functional state of a user of a vehicle according to an embodiment of the present disclosure;

FIG. 3 illustrates a flow chart of a method for determining a functional state of a user of a vehicle according to an embodiment of the present disclosure;

FIG. 4 illustrates a flow chart of a method for determining a functional state of a user of a vehicle according to another embodiment of the present disclosure;

FIG. 5 illustrates a flow chart of a method for determining a functional state of a user of a vehicle according to yet another embodiment of the present disclosure; and

FIG. 6 illustrates a flow chart of a method for training a neural network according to another embodiment of the present disclosure.

### DETAILED DESCRIPTION OF EXEMPLARY EMBODIMENTS

The present disclosure provides a method and system for detecting human functional states. Multiple human state components, also referred to as conditions, are identified simultaneously and consistently. In illustrated, exemplary embodiments, a plurality of interrelated conditions of a user of a vehicle is detected and monitored. Example conditions are stress and fatigue. The conditions are processed to determine the user's functional state. The functional state can indicate if the user is fit to operate the vehicle. By taking into account a combination of interrelated conditions, individual conditions can be detected more accurately, and the user's state can be determined more reliably.

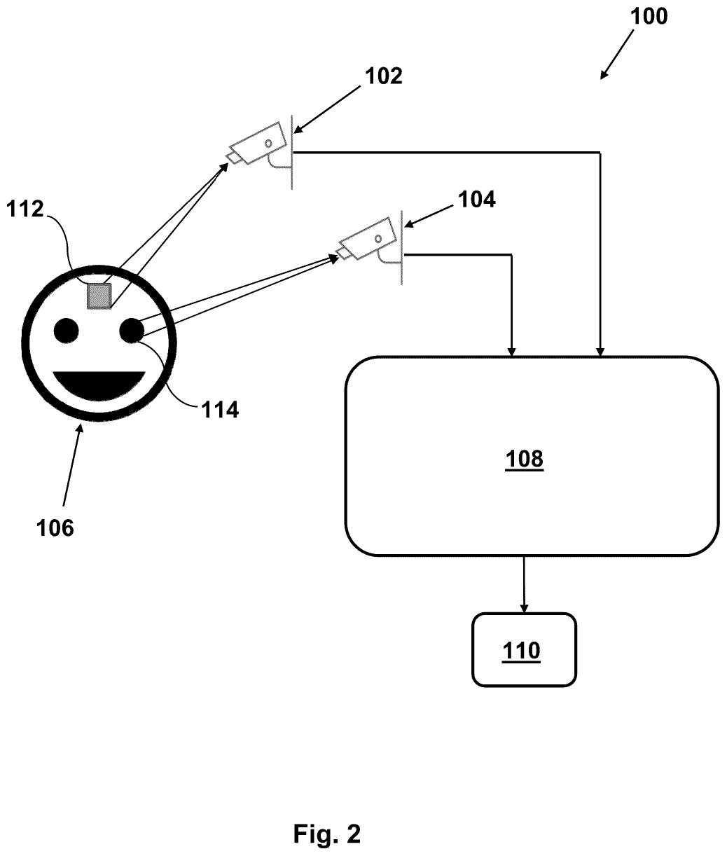

With reference to FIG. 2, a system 100 according to an embodiment includes first and second sensors 102 and 104. In the illustrated embodiment, the sensors 102 and 104 are cameras. The cameras 102 and 104 may be installed in a vehicle operated by a user 106. The vehicle may be a car, although the present disclosure is not limited in this regard. The cameras 102 and 104 are arranged to be directed at the user's face to detect light reflected from the user's face. The cameras 102 and 104 generate output signals representative of the detected light. The output signals are transmitted to a processing device 108 communicatively coupled to the cameras 102 and 104. The processing device 108 may be arranged in the vehicle, e.g. the head unit of a car. Alternatively, the processing device 108 may be arranged remotely from the vehicle, e.g. at a remote control station. In another alternative, the processing device 108 may include several processing elements, some or all of which may be arranged in the vehicle or remotely. In any case, the processing device 108 is configured to process the signals received from the cameras 102 and 104 to determine conditions and an overall state of the user 106, as described in more detail below. Following completion of the processing, an output signal is generated and transmitted to an interface 110. The interface 110 may be arranged to transmit the output signal to another entity, e.g. to a display in the vehicle, thereby to enable displaying the determined state of the user or associated information. Alternatively, the output signal can be transmitted to a control unit of the vehicle, thereby enabling an intervention in the operation of the vehicle.

The first camera 102 may be arranged to detect light reflected from a selected skin patch 112 on the user's face. The skin patch 112 may be located on the user's forehead or one of the user's cheeks. Light reflected from such skin patch is particularly suited for rPPG processing by the processing device 108. RPPG processing includes detecting changes in corresponding pixels of consecutive image frames, for example changes in color and/or intensity. Such changes may be caused by momentary changes in the blood flow underneath the skin patch 112. This may provide information about heartbeat parameters of the user 106 including, but not limited to heartbeat rate, heartbeat strength, heartbeat rhythm and inter-beat intervals.

The second camera 104 may be arranged to record a series of images of an eye region 114 that contains one of the eyes of the user. Output signals of the camera 104 are transmitted to the processing device 108. The processing device 108 processes the received signals to determine selected eye parameters such as eye or eyelid movements, eye gaze and eyelid positions. These eye parameters also are indicative of a condition of the user such as fatigue, stress and distraction.

The cameras 102 and 104 may have RGB sensors capable of capturing a series of image frames, using the visible light spectrum, with a resolution sufficient to allow facial contours and features, such as locations of the eyes, eyebrows, nose, and mouth, to be ascertained. Each pixel can have associated red-green-blue (RGB) values. Capturing visible light, and thereby allowing RGB values to be associated with the pixels, allows the color component which is best suited for rPPG and further analysis to be selected. An optional light source (not shown) may be added to provide artificial light and illuminate the face of the user 106, thereby enabling a more reliable feature detection, for example when there is fluctuating or insufficient natural light.

In another embodiment, one or both of the cameras 102 and 104 are infrared cameras. In this embodiment, the system 100 may also include an infrared light source (not shown) to illuminate the face of the user 106 with infrared light. Using infrared imaging and infrared illumination may enable more a reliable feature detection at night or in other situations when natural light is absent or insufficient.

The cameras 102 and 104 may be self-adjusting. In particular, the cameras 102 and 104 may be configured to detect facial features and adjust camera parameters such as orientation, focusing, aperture etc. to be optimized for the purposes of detecting light from the face of the user 106.

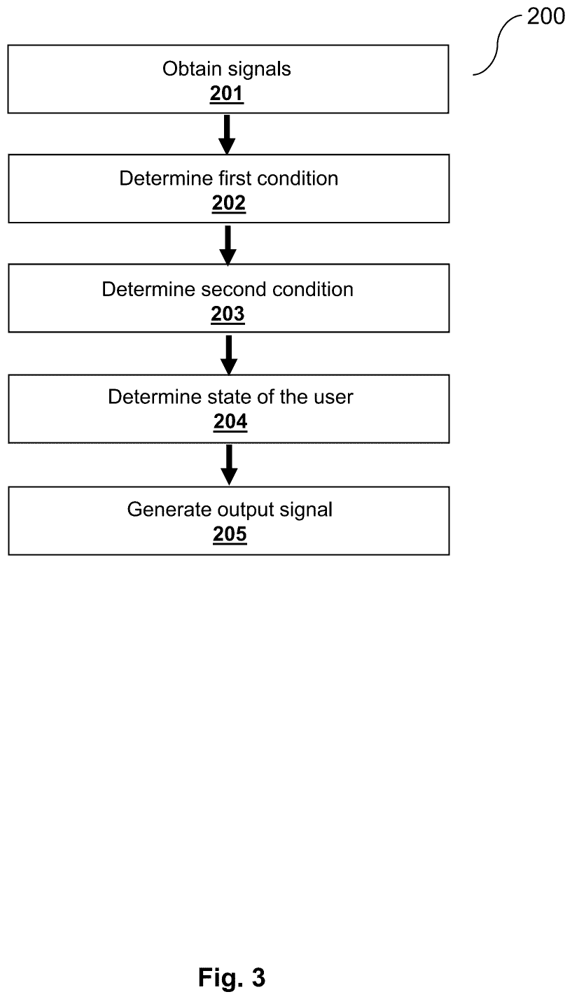

FIG. 3 illustrates a method 200 according to an embodiment of the present disclosure. The method can be performed by the processing device 108 of FIG. 2. In step 201, signals are obtained from a plurality of sensors in the vehicle, e.g. from the cameras 102 and 104. Accordingly, the signals may be image signals representing light reflected from the skin patch 112 and the eye region 114 of the user 106. The signals contain information representative of different physiological parameters of the user 106, for example heartbeat and eye parameters. In steps 202 and 203, this information is processed to determine at least first and second conditions of the user 106. For example, step 202 may include extracting and processing heartbeat parameters to determine a heartbeat rate. The heartbeat rate may correspond to stress level of the user. For example, an increased heartbeat rate indicates an increased stress level. This corresponds to the first condition. Step 203 may include extracting and processing eye parameters to determine a second condition of the user 106. For example, the eye parameter may correspond to a rate of eye movements. A high rate of eye movements may also indicate an increased stress level. This corresponds to the second condition. Accordingly, in this example, the first and second conditions correspond to one another (“increased stress level”). In step 204, the first and second conditions are processed to determine the user's overall state. In the present example, the first and second conditions are identical, and the user's state may be determined to correspond to the first and second conditions, namely a state corresponding to “increased stress level”. In step 205, the state determined in step 204 may be used to generate an output signal. For example, a state corresponding to “increased stress level” may cause an output signal that is used to intervene in the operation of the vehicle. For example, the output signal may be used to activate a speed limiter to prevent the user from driving above a certain speed.

Accordingly, the user's state is determined based upon at least two conditions. If the conditions are consistent with one another, the user's state can be determined with greater confidence. This, in turn, enables taking actions such as intervening in the vehicle's operation that would otherwise not be recommendable. Furthermore, in an embodiment, it is possible define different levels of confidence for different actions. For example, intervening in the operation of the vehicle may require at least three consistent conditions to be present. In particular, this may require three different physiological parameters to indicate the same condition such as “increased stress level”. This could be a high heart beat rate, rapid eye movements, and rapid eyelid movements.

In another example, if a normal stress level is determined as the user's state, no output signal is generated. In yet another example, if the first and second conditions are inconsistent with one another, the state of the user may be determined to be inconclusive, and the output signal could be an error signal.

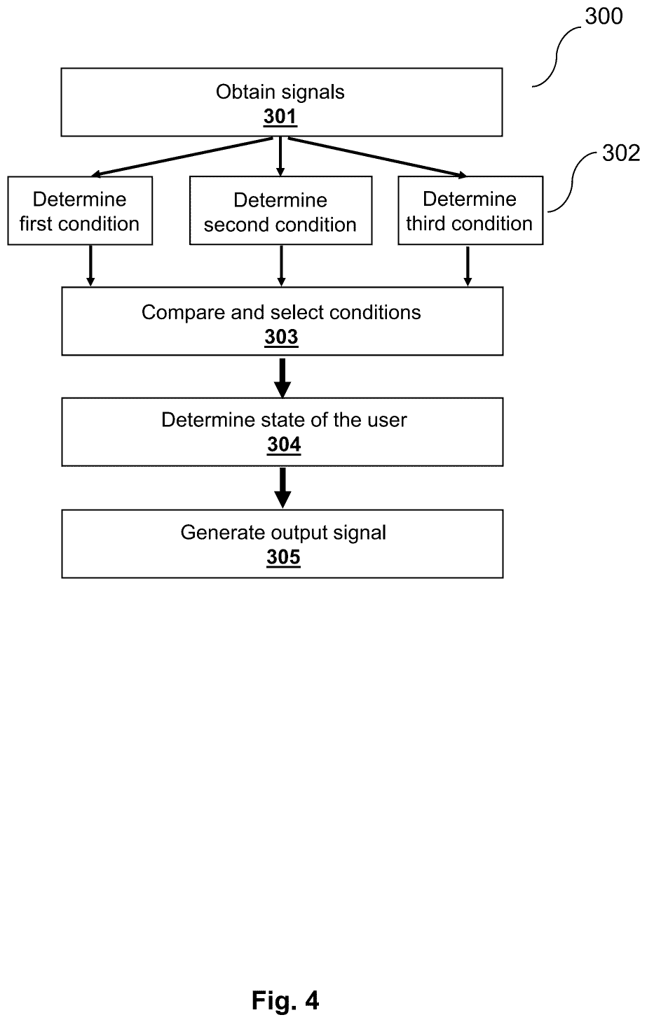

FIG. 4 illustrates a method 300 according to another embodiment. Method 300 can also be performed by the processing device 108 of FIG. 2. In this embodiment, the user's state is determined based on selecting and/or combining individual conditions of the user. In step 301, signals from a plurality of sensors are obtained. Step 301 may be identical to step 201 in FIG. 2. In step 302, at least three conditions are determined. Each condition may be determined based on different physiological parameters from the other conditions. For example, the first condition may be determined based on a heartbeat rate parameter. The second condition may be determined based on an eye movement parameter. The third condition may be determined based on an eyelid movement parameter. In step 303, the first, second and third conditions are compared with one another. If all three conditions are found to be consistent with one another, the method proceeds to step 304 and determines the user's state based on all three conditions. However, if in step 303 one of the three conditions is found to be inconsistent with the other two conditions, then the other two conditions are selected for determining the user's state in step 304. Accordingly, the present embodiment provides some redundancy and increases the consistency in the determination of the user's state over longer periods of time. In step 305, an output signal is generated based on the determined user state. Step 305 may be identical to step 205 in FIG. 3.

Method 300 is an illustrative example, and the present disclosure is not limited in this regard. For example, there could be any number of conditions, based on different physiological parameters derived from the same or different sensor signals. Also, it is possible to use the same parameters for some or all of the conditions, but derived from different signals or sensors. Moreover, it is possible to begin by determining a first number of conditions, and then proceeding to determine a second number of conditions only if at least one of the first number of conditions is found to be inconsistent with the other ones of the first number of conditions.

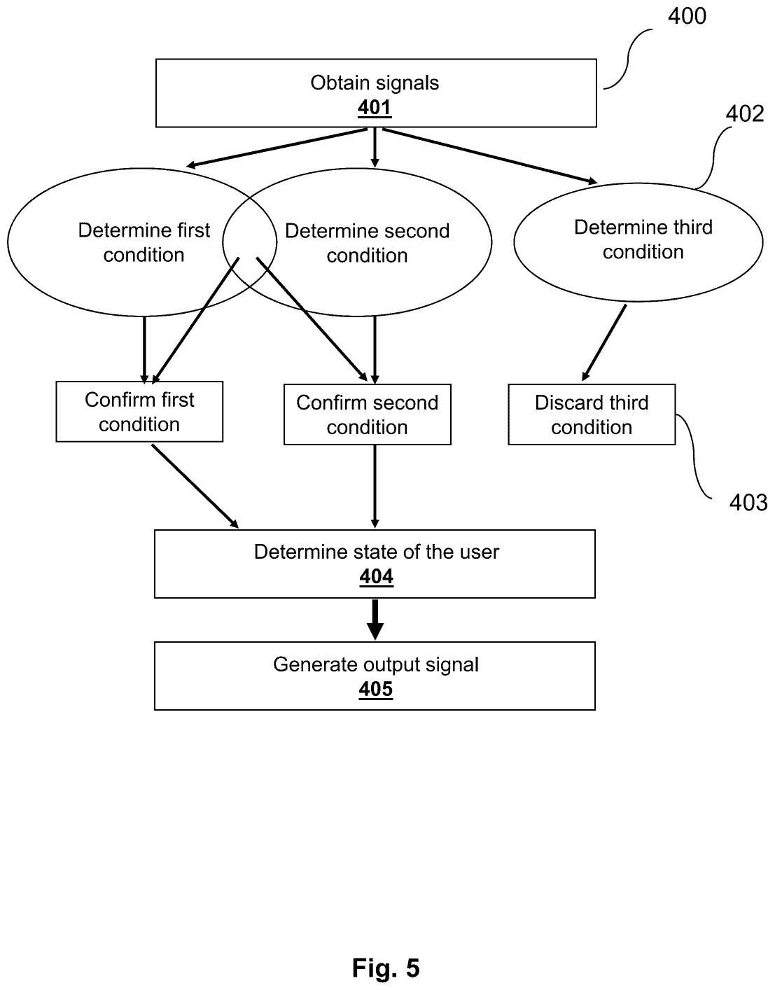

FIG. 5 illustrates a method 400 according to another embodiment. In this embodiment, the interdependency between conditions is taken into account to improve the detection of individual conditions and refine the determination of the user's overall state. In step 401, signals from a plurality of sensors are obtained. Step 401 may be identical to step 301 in FIG. 4. In step 402, at least three conditions are determined. Each condition may be determined based on different physiological parameters, as explained in connection with FIG. 4. The conditions may be interrelated, as schematically illustrated by the overlapping ellipses for the first and second conditions. For example, a detection of the first condition may be more likely correct if the second condition is detected at the same time. Similarly, a detection of the third condition may be less likely correct if both the first and second conditions are detected at the same time. Accordingly, the interrelation between different conditions may be used to verify the correct detection of individual conditions.

In FIG. 5, only three conditions are shown for simplicity. However, the present disclosure is not limited in this regard. There can be multiple conditions, some of which “overlap” with one another, while others exclude one another. The presence of a combination of conditions may make the presence of certain individual conditions more or less likely. Moreover, based on these interrelations, the conditions, in their entirety, form a pattern that indicates an overall state of the user. Different such patterns may be associated with different user states.

In step 403, the knowledge of these interrelations is used to verify the results of step 402. In the illustrated example, the detection of the first and second conditions is confirmed, whereas the detection of the third condition is discarded. Thus, in the illustrated example, only two conditions are selected, but these two conditions can be used to determine the user's overall state with more confidence.

In step 404, the conditions selected in step 403 are processed to determine the user's state. In step 405, an output signal is generated based on the user state. Steps 404 and 405 may be identical to steps 304 and 305 in FIG. 3.

In an embodiment, the interrelations between conditions can be used to determine probabilities of correctly detecting individual conditions. Each pattern consisting of a set of detected conditions can indicate a probability for the presence of an individual condition. For example, with reference to FIG. 5, if the first and second conditions are detected at the same time, the probability of correctness of the detection of the first condition could be set to 80%. If, at the same time, the third condition is also detected, which is considered inconsistent with the first and second conditions, the probability of correctness associated with the first condition could be reduced to 70%. These values represent illustrative, non-limiting examples.

In another example, again with reference to FIG. 5, step 403 could be part of an iterative process. For any given combination of multiple detected conditions, the one that is determined to be least likely can be discarded. The resulting new combination of conditions can undergo the same determination and selection. This can be repeated until a predetermined threshold is reached. The threshold can be implemented in terms of a number of remaining conditions, probabilities of individual remaining conditions, a probability of the combination of remaining conditions, or suchlike.

The selection of conditions in step 403 can be implemented by neural networks. Such neural network can be trained based on different patterns of conditions and how they are interrelated with individual conditions. The same applies, in principle, to the determination of the user state in step 404. To this end, a neural network can be trained based on different combinations of conditions and their association with overall user states.

More particularly, in an embodiment, machine learning (“ML”) is used to implement a detector that defines the relationships between physiological parameters or biosignals and individual components of the user's overall state. Such detector is also referred to as “ML detector” herein and can be implemented by a neural network that is trained to learn these relationships. Generally, training data indicating such relationships can be obtained through experiment, during normal operation of a system implementing the method, or simply based on experience.

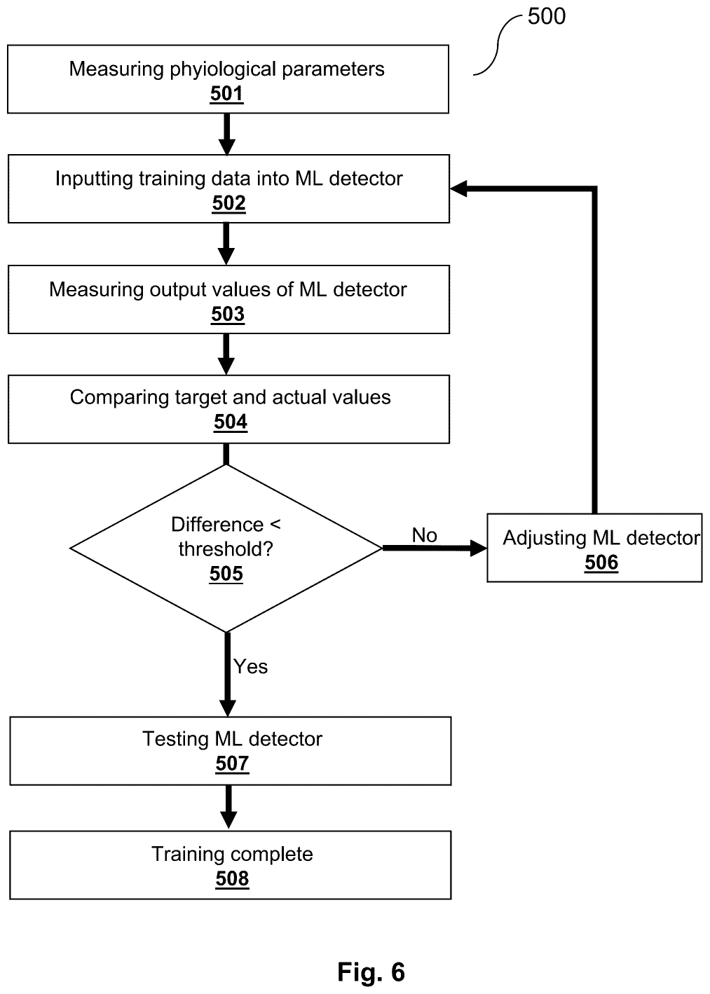

An exemplary training process of an ML detector is illustrated in FIG. 6. The ML detector is trained using known or assumed relationships between physiological parameters and psychophysiological conditions of the user. For example, a person who is solving a complex mathematical problem can be assumed to experience a high degree of cognitive load. Accordingly, in step 501, physiological parameters are measured while the user is solving a complex problem, e.g. a mathematical problem. In step 502, the measured values are input in the ML detector as training data. Inputting such data into the ML detector is expected to result in an output indicating a high degree of cognitive load. The expected output of the ML detector corresponds to “target values”. In step 503, actual values output by the ML detector are measured and, in step 504, these measured values are compared with the target values. If, in step 505, the difference between the target values and the measured values is found to be larger than a predetermined threshold, the process proceeds to step 506. In step 506, a so-called error function is generated to identify the measure of deviation between the actual and expected behavior of the ML detector for a particular instance of physiological parameters. The error function is used to adjust the “behavior” of the ML detector to reduce the discrepancy between target and actual values. For example, the error function is used to adjust neural network weights. The process of adjusting weights in a neural network is generally known as back propagation.

Steps 502-506 can be repeated until a stop condition is fulfilled. There can be different stop conditions. In one example, the stop condition is fulfilled when the discrepancy between target and actual values is below a predetermined threshold. In another example, the stop condition is fulfilled when the accuracy of the ML detector stops increasing, or if it is increasing by amounts below a threshold. In a further example, steps 502-506 are repeated for a predetermined amount of adjustment iterations, e.g., until a predetermined “budget” of iterations available for training the ML detector is spent. In any case, when the stop condition is fulfilled in step 505, the process proceeds to step 507. Step 507 is an optional step to test the accuracy and reliability of the ML detector using test data sets. The test data sets should adequately represent practically significant scenarios when using the ML detector in a “real life” application. If the accuracy achieved during the training (with or without step 507) is considered satisfactory, the training process may be terminated (step 508). Otherwise, it may be repeated using different training data. Thereafter, the ML detector is ready for use to identify human functional states under real-life conditions.

In the following, a more specific, illustrative example of training the ML detector is described. The training process can follow the steps of FIG. 6. In this example, the ML detector implements a two-component cognitive load detector for use in a vehicle. The ML detector is to detect and indicate if the driver experiences cognitive load, and whether or not the cognitive load is related to driving. Suitable training data is generated based on the following protocol including several stages. The protocol can be implemented during real-life driving or in a driving simulator. In each stage, physiological parameters (biosignals) of the user are measured while the indicated requirements are met. The measured values are then used as input (training) data of the ML detector. In each stage, and for each requirement, an expected output (target value) for a corresponding condition is known.

### Stage 1:

Requirements: There is no complex driving-related task. (The term “complex” is intended to indicate a task that clearly goes beyond ordinary driving situations.). There is no additional non-driving-related task.
Target values: The ML detector is to output the values 0 for “driving tasks” and 0 for “non-driving tasks”.

### Stage 2:

Requirements: There is no complex driving-related task. There is an additional non-driving-related task.
Target values: The ML detector is to output the values 0 for “driving tasks” and 1 for “non-driving tasks”.

### Stage 3:

Requirements: There is a complex driving-related task. There is no additional non-driving-related task.
Target values: The ML detector is to output the values 1 for “driving tasks” and 0 for “non-driving tasks”.

### Stage 4:

Requirements: There is a complex driving-related task. There is an additional non-driving-related task.
Target values: The ML detector is to output the values 1 for “driving tasks” and 1 for “non-driving tasks”.

After training, the ML detector, when fed with data representing physiological parameters obtained in real-life driving situations, can generate output signals indicating the presence or absence of high cognitive load related or unrelated to driving:

Outputs for driving-related task:
0: indicates no complex driving-related task is being solved by the subject. (However, the driver may be experiencing a low level of cognitive load associated with ordinary driving tasks.)
1: indicates that the user is experiencing a high level of cognitive load due to a complex driving-related task.

Outputs for non-driving-related task:
0: the driver is not experiencing a significant cognitive load due to a non-driving-related task.
1: the driver is experiencing a significant cognitive load due to a non-driving-related task.

The following is an analogous example of an ML detector implementing a three-component cognitive load detector, also for use in a vehicle. In this example, it is additionally possible to identify a level of the driver's overall cognitive load. Training of the three-component cognitive load detector can be performed using the following protocol:

### Stage 1:

Requirements: There is no complex driving-related task. There is no additional non-driving-related task.
Target values: The ML detector is to output the values 0 for “driving tasks”, 0 for “non-driving tasks”and 0 for “overall load”.

### Stage 2:

Requirements: There is no complex driving-related task. There is an additional non-driving-related task.
Target values: The ML detector is to output the values 0 for “driving tasks”, 1 for “non-driving tasks”and 1 for “overall load”.

### Stage 3:

Requirements: There is a complex driving-related task. There is no additional non-driving-related task.
Target values: The ML detector is to output the values 1 for “driving tasks”, 0 for “non-driving tasks”and 1 for “overall load”.

Stage 4:
Requirements: There is a complex driving-related task. There is an additional non-driving-related task.
Target values: The ML detector is to output the values 1 for “driving tasks”, 1 for “non-driving tasks”and 2 for “overall load”.

After training, the ML detector, when fed with data representing physiological parameters obtained in real-life driving situations, can generate output signals indicating the presence or absence of high cognitive load related or unrelated to driving. These outputs are identical to the outputs of the two-component cognitive load detector above. In addition, the ML detector generates an output indicating a level of the driver's overall cognitive load:
0: low overall load.
1: medium overall load.
2: high overall load.

In another example, the ML detector can implement a three-component cognitive load detector similar to the one described above but trained to determine the driver's overall cognitive load over a broader range of states. Training of this three-component cognitive load detector can be performed using the following protocol:

### Stage 1:

Requirements: There is no complex driving-related task. There is no additional non-driving-related task.
Target values: The ML detector is to output the values 0 for “driving tasks”, 0 for “non-driving tasks” and 1 for “overall load”. The value 1 for “overall load” indicates a base level of cognitive load in ordinary driving situations, without distraction resulting from non-driving-related tasks.

### Stage 2:

Requirements: There is no complex driving-related task. There is an additional non-driving-related task.
Target values: The ML detector is to output the values 0 for “driving tasks”, 1 for “non-driving tasks”and 2 for “overall load”.

### Stage 3:

Requirements: There is a complex driving-related task. There is no additional non-driving-related task.
Target values: The ML detector is to output the values 1 for “driving tasks”, 0 for “non-driving tasks”and 2 for “overall load”.

### Stage 4:

Requirements: There is a complex driving-related task. There is an additional non-driving-related task.
Target values: The ML detector is to output the values 1 for “driving tasks”, 1 for “non-driving tasks”and 3 for “overall load”.

### Stage 5:

Requirements: There is a very low level of overall activity due to (e.g.) sleepiness.
Target values: The ML detector is to output no values (or “not defined”) for “driving tasks”and “non-driving tasks”, and 0 for “overall load”.

After training, the ML detector, when fed with data representing physiological parameters obtained in real-life driving situations, can generate output signals corresponding to those of the three-component cognitive load detector above. However, in this example, the output indicating the driver's overall cognitive load has a higher resolution:
0: very low overall load.
1: low overall load.
2: medium overall load
3: high overall load.

The above represents a simple, illustrative example of how different components of a driver's state can be combined to determine whether the driver is in a state fit do drive. An output of 0 or 3 for the overall load can be considered critical and used to cause an intervention in the operation of the vehicle, as described above. An output of 1 or 2 can be considered to correspond to a “healthy”, i.e. safe driving state.

As described above, the input signals of the ML detector represent one or more physiological parameters indicating different components of a user's state. The input signals may be obtained from sensors such as the cameras 102 and 104 in FIG. 2. The input signals may represent a single parameter from which a plurality of components of the user's state can be derived. Alternatively, the input signals may represent a combination of different physiological parameters, such as eye movement, eye closure, gaze direction, and combinations thereof; heartbeat parameters, e.g. one ore more of pulse waves, IBI (interbeat intervals), HR (heart rate), HRV(heart rate variability) and their derivatives; respiratory information such as frequency, respiratory patterns; muscle activity, temperature, pressure, oxygen saturation; etc.

Such physiological parameters can be derived from various physical signals in active and passive sensing systems, for example video data from IR or visible-range cameras, with or without active backlight; reflected radar signals at different frequencies and on different construction principles (pulse, constant radiation, beam pattern controlled, etc.); voltages derived from contact sensors related to muscle activity (different muscle groups); heartbeat activity (ECG); brain activity (EEG); pressure sensors, compression sensors, displacement sensors and the like that can be attached or linked to the human body; etc.

The components of the user's overall state can represent different emotions, and combinations thereof, that affect one or more measurable physiological parameters. Such emotions can include anger, disgust, fear, joy, sadness, surprise, indifference, etc. Such emotions can be described or measured in different ways, e.g. based on sensory activity (including subtypes, such as visual, auditory, tactile, odors, vestibular, gustatory); motor activity (including subtypes); cognitive processes (including subtypes, such as memory, attention, thinking); emotional processes (including subtypes); etc.

The present disclosure is not limited to any particular types of components. However, any component should be detectable, e.g. by way of measuring physiological parameters. In particular, each component should be reproducible in training or experimental setups, to enable an interpretation of measurements of associated physiological parameters.

The above approach above has been described in connection with state detectors, but it can also be used as a tool in data mining to extract and identify psychophysiological states of a person, on the basis of data describing physiological parameters of that person.

The description of embodiments and aspects has been presented merely for purposes of illustration and explanation. Suitable modifications and variations to the embodiments and aspects may be performed in light of the above, and different embodiments and aspects may be combined where possible and appropriate, without departing from the scope of protection as determined by the claims.

1. A computer-implemented method for detecting functional states of a user, the method comprising:
obtaining one or more signals from one or more sensors, wherein the signals are representative of a plurality of physiological parameters of the user;
determining a plurality of conditions associated with the user based on values of the physiological parameters;
determining a functional state of the user based on a combination of the conditions; and
generating an output signal to indicate the functional state of the user.

2. The computer-implemented method of claim 1, further comprising:
determining at least one of the conditions based on at least one other of the conditions.

3. The computer-implemented method of claim 2, further comprising:
determining at least one of the conditions based on a combination or pattern of other ones of the conditions.

4. The computer-implemented method of claim 1, wherein each of the conditions is representative of a level of cognitive load, stress or drowsiness of the user, and/or wherein the functional state of the user is indicative of the user's fitness to operate, in particular drive a vehicle.

5. The computer-implemented method of claim 4, wherein the cognitive load includes cognitive load related and/or unrelated to operating, in particular driving the vehicle by the user.

6. The computer-implemented method of claim 1, wherein determining the functional state of the user comprises selecting one of a plurality of predetermined states, wherein each of the plurality of predetermined states is associated with a respective combination or pattern of the conditions.

7. The computer-implemented method of claim 1, further comprising:
determining that some of the conditions are mutually exclusive; and
discarding one or more or all of the mutually exclusive conditions when determining the functional state of the user.

8. The computer-implemented method of claim 1, wherein the method is implemented in a vehicle, and wherein generating an output signal to indicate the functional state of the user comprises one or more of:
alerting the user of the vehicle;
controlling the vehicle, in particular adjusting a user interface in the vehicle or adjusting one or more vehicle control systems;
communicating the functional state of the user to another vehicle or an external server or control station.

9. The computer-implemented method of claim 1, wherein the one or more physiological parameters include:
one or more heartbeat parameters, in particular a heartbeat rate, a heartbeat rate variability, and/or a heart interbeat interval; and/or
one or more eye parameters, in particular an eye movement, an eyelid movement, an eye position, an eyelid position, and/or an eye gaze.

10. A system for detecting functional states of a user, the system comprising:
one or more sensors to detect one or more signals, wherein the signals are representative of a plurality of physiological parameters of the user; and
at least one processing device coupled to the sensors and configured to:
determine a plurality of conditions associated with the user based on values of the physiological parameters;
determine a functional state of the user based on a combination of the conditions; and
generate an output signal to indicate the functional state of the user.

11. The system of claim 10, wherein the processing device is configured to:
determine at least one of the conditions based on at least one other of the conditions, and in particularly based on a combination or pattern of other ones of the conditions.

12. The system of claim 10, wherein the processing device is configured to determine the functional state of the user by selecting one of a plurality of predetermined states, wherein each of the plurality of predetermined states is associated with a respective combination of the conditions.

13. The system of claim 10, wherein the processing device is configured to:
determine that some of the conditions are mutually exclusive; and
discard one or more or all of the mutually exclusive conditions when determining the functional state of the user.

14. The system of claim 10, wherein the system is an in-vehicle system, and wherein generating an output signal to indicate the functional state of the user comprises one or more of:
alerting the user of the vehicle;
controlling the vehicle, in particular adjusting a user interface in the vehicle or adjusting one or more vehicle control systems;
communicating the functional state of the user to another vehicle or an external server or control station.

15. The system of claim 10, wherein the system is an in-vehicle system, and/or wherein the one or more sensors comprise one or more image sensors, in particular an RGB and/or an infrared camera.

16. A system for detecting functional states of a user, the system comprising:
one or more sensors to detect one or more signals, wherein the signals are representative of a plurality of physiological parameters of the user; and
at least one processing device coupled to the sensors and configured to:
determine a plurality of conditions associated with the user based on values of the physiological parameters;
determine a functional state of the user based on a combination of the conditions; and
generate an output signal to indicate the functional state of the user, wherein the processing device is configured to:
determine at least one of the conditions based on at least one other of the conditions, and in particularly based on a combination or pattern of other ones of the conditions, wherein the processing device is further configured to determine the functional state of the user by selecting one of a plurality of predetermined states, wherein each of the plurality of predetermined states is associated with a respective combination of the conditions.

17. The system of claim 16, wherein the processing device is configured to:
determine that some of the conditions are mutually exclusive; and
discard one or more or all of the mutually exclusive conditions when determining the functional state of the user.

18. The system of claim 17, wherein the system is an in-vehicle system, and wherein generating an output signal to indicate the functional state of the user comprises one or more of:
alerting the user of the vehicle;
controlling the vehicle, in particular adjusting a user interface in the vehicle or adjusting one or more vehicle control systems;
communicating the functional state of the user to another vehicle or an external server or control station.

19. The system of claim 17, wherein the system is an in-vehicle system, and/or wherein the one or more sensors comprise one or more image sensors, in particular an RGB and/or an infrared camera.
